(19) United States
(12) Patent Application Publication
(10) Pub. No.: US 2002/0052615 A1
(43) Pub. Date: May 2, 2002

(54) Cutting blade assembly for a microkeratome

(76) Inventors: Rod Ross, Mission Viejo, CA (US); Greg Hughes, Mission Viejo, CA (US); Mark Moyer, San Marcos, CA (US); J. R. Dennewill, Cerritos, CA (US)

Correspondence Address: IRELL & MANELLA LLP, 840 NEWPORT CENTER DRIVE, SUITE 400, NEWPORT BEACH, CA, 92660, US

(21) Appl. No.: 09/895,304

(22) Filed: Jun. 29, 2001

Related U.S. Application Data
(63) Continuation-in-part of application No. 09/585,566, filed on Jun. 2, 2000.

(51) Int. Cl. A61F 9/00

(52) U.S. Cl. 606/166; 606/172

(57) ABSTRACT

A blade assembly that can be assembled into a microkeratome which is used to cut a cornea. The blade assembly is constructed in a manner that minimizes the tolerance of the cutting depth into the cornea. The blade assembly includes a blade holder that can be pressed onto a blade. The relative position of the blade holder and the blade can be established with a tool assembly that accurately controls the distance between a reference surface of the blade holder and the cutting edge of the blade. This distance defines the cutting depth of the blade. The tool allows a manufacturer to closely control the cutting depth of the blade assembly. The blade holder may have a color or other indicator that provides an indication of the cutting depth of the blade assembly. The blade assembly may be carried in a package that has an opening to allow visual inspection of the blade. The package may have a color or other indicator that provides a visual indication of the blade cutting depth. The blade holder may be constructed from a molded plastic material and contain a number of cavities that minimize warpage of the plastic holder. The plastic holder may also a hole to allow a bonding agent to be applied during the assembly process to bond the blade holder to the blade.

### REFERENCE TO CROSS-RELATED APPLICATION

This application is a continuation-in-part of application Ser. No. 09/585,566 filed on Jun. 2, 2000, pending.

### BACKGROUND OF THE INVENTION

1. Field of the Invention

The present invention relates to a blade assembly that can be assembled into a medical device that is used to cut a cornea.

2. Background Information

There have been developed a number of different surgical techniques to correct hyperopic or myopic conditions of a human eye. U.S. Pat. No. 4,840,175 issued to Peyman discloses a procedure wherein a thin layer of the cornea tissue is cut and removed from the cornea. A laser beam is then directed onto the exposed corneal tissue in a predetermined pattern. The laser beam ablates corneal tissue and changes the curvature of the eye. This procedure is sometimes referred to as Laser in situ Keratomileusis (LASIK).

U.S. Pat. No. Re 35,421 issued to Ruiz et al. discloses a device for cutting a cornea in a LASIK procedure. Such a device is commonly referred to as a microkeratome. The Ruiz microkeratome includes a ring that is placed onto a cornea and a blade that is located within an opening of the ring. The device also contains a drive mechanism which moves the blade across the cornea in a first direction while the blade moves in a reciprocating transverse direction to cut the eye. The device can create a lamella flap of the cornea which is flipped back so that the stromal bed of the cornea can be ablated with a laser.

U.S. Pat. No. 6,051,009 issued to Hellenkamp et al. discloses a microkeratome that is sold under the trademark HANSATOME. The HANSATOME microkeratome moves the blade in an arcuate path about the cornea. The HANSATOME includes a disposable blade assembly that can be readily loaded and removed from the device. The blade assembly includes a blade holder that is attached to a cutting blade. The blade holder has a recess that receives the end of a drive shaft. Rotation of the output shaft both moves the blade in an arcuate path and moves the blade in a back and forth motion to create the lamella flap of the cornea.

It is critical to control the depth of the cut to prevent a deep or shallow cut of the cornea. The depth of the cut is a function of the distance between the cutting edge of the blade and a reference surface of the blade holder. The HANSATOME blade holder is attached to the cutting blade by a pair of plastic protrusions that extend from the blade holder through corresponding apertures of the blade. The plastic protrusions located on the underside of the blade holder are then ultrasonically welded to the top side of the blade.

The accuracy of the distance between the cutting edge and the reference surface, and thus the depth of the cut into the cornea, is dependent upon the mechanical tolerance between the cutting edge and the aperture of the blade, and the mechanical tolerance between the protrusions and the reference surface of the blade holder. This tolerance “build up” can reduce the predictability of the cutting depth. It would be desirable to provide a blade assembly and process for assembling the blade assembly that would tightly control the tolerance between the cutting edge and the reference surface and thus the depth of the cut.

### BRIEF SUMMARY OF THE INVENTION

A blade assembly that includes a blade holder coupled to a blade. The blade holder has a color or other visual indicator indicative of a cutting depth of the blade.

### BRIEF DESCRIPTION OF THE DRAWINGS

FIG. 1 is a side view of an embodiment of a microkeratome with a blade assembly;

FIG. 2 is an exploded top view of an embodiment of a blade assembly;

FIG. 3 is a back view of a blade holder of the blade assembly;

FIG. 4 is a back view of the blade assembly;

FIG. 5 is an exploded top view of another embodiment of a blade assembly;

FIG. 6 is a back view of a blade holder of the assembly shown in FIG. 5;

FIG. 7 is a back view of the blade assembly shown in FIG. 5;

FIG. 8 is a perspective view of another embodiment of a blade assembly;

FIG. 9 is a side view of the blade assembly shown in FIG. 8;

FIG. 10 is front view of the blade assembly shown in FIG. 8;

FIG. 11 is a side view of another embodiment of a blade assembly;

FIG. 12 is a top view showing a blade holder and a blade secured by a stabilizing post that is used to calibrate the holder;

FIG. 13 is a side view showing the blade holder assembled to the blade;

FIG. 14 is a top view showing a blade secured by a clamp that is used to calibrate the blade holder;

FIG. 15 is a side view of another embodiment of a blade assembly;

FIG. 16 is a side view of another embodiment of a blade assembly;

FIG. 17 is a side view of the blade assembly shown in FIG. 16;

FIG. 18 is rear perspective view of an alternate embodiment of a blade assembly;

FIG. 19 is an exploded view of the blade assembly;

FIG. 20 is a front view of a blade assembly package;

FIG. 21 is a side view of the blade assembly package;

FIG. 22 is a perspective view of a tool assembly used to assemble a blade assembly;

FIG. 23 is a sectional view of the tool assembly;

FIG. 24 is a side view of a tool gauge used to determine whether the blade holder is within manufacturing tolerances;

FIG. 25 is a top view of alternate embodiment of a blade assembly;

FIG. 26 is a top view of a caliper assembly.

### DETAILED DESCRIPTION OF THE PREFERRED EMBODIMENT

A blade assembly that can be assembled into a microkeratome which is used to cut a cornea. The blade assembly is constructed in a manner that minimizes the tolerance of the cutting depth into the cornea. The blade assembly includes a blade holder that can be pressed onto a blade. The relative position of the blade holder and the blade can be established with a tool assembly that accurately controls the distance between a reference surface of the blade holder and the cutting edge of the blade. This distance defines the cutting depth of the blade. The tool allows a manufacturer to closely control the cutting depth of the blade assembly.

The blade holder may have a color or other indicator that provides an indication of the cutting depth of the blade assembly. The blade assembly may be carried in a package that has an opening to allow visual inspection of the blade. The package may have a color or other indicator that provides a visual indication of the blade cutting depth. The blade holder may be constructed from a molded plastic material and contain a number of cavities that minimize warpage of the holder. The plastic holder may also have a hole to allow a bonding agent to be applied during the assembly process to bond the blade holder to the blade.

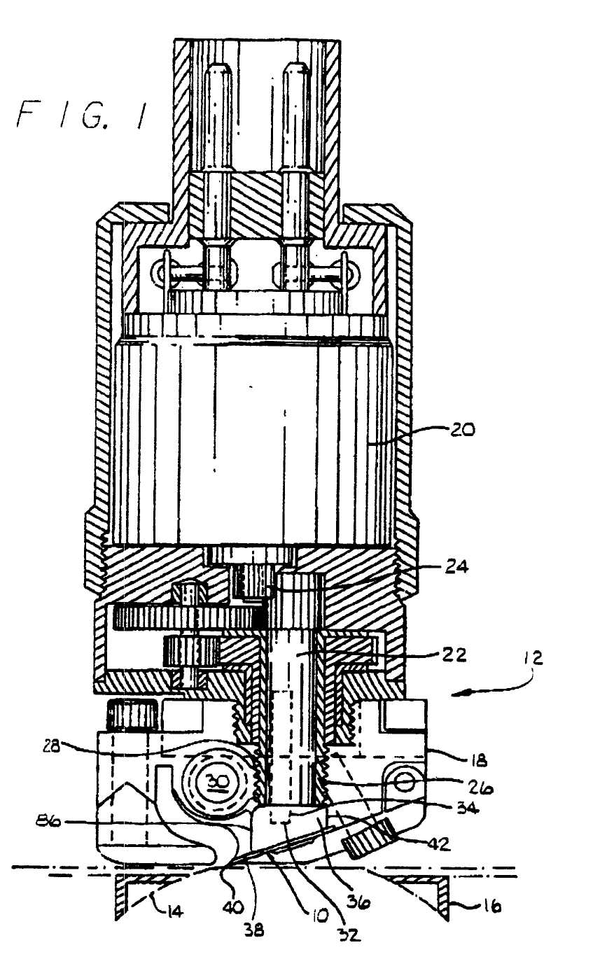

Referring to the drawings more particularly by reference numbers, FIG. 1 shows an embodiment of a blade assembly 10 assembled into a microkeratome 12. The microkeratome 12 is typically used to create a lamella in a cornea 14 as an initial step in a LASIK procedure. The microkeratome 12 may be the same or similar to the device disclosed in U.S. Pat. No. 6,051,009 issued to Hellenkamp et al., which is hereby incorporated by reference. The device disclosed in the '009 patent is also sold by Bausch Lomb under the trademark HANSATOME. Although the HANSATOME is shown and described, it is to be understood that the blade assembly 10 of the present invention can be used in other microkeratomes.

The microkeratome 12 includes a ring 16 that is placed onto the cornea 14 and typically held in place by a vacuum pressure. The microkeratome 12 also includes a cutting head assembly 18 that is coupled to the ring 16. The cutting head assembly 18 includes a motor 20 that is coupled to an output shaft 22 by a gear assembly 24. The output shaft 22 has an external thread 26 that is coupled to a corresponding thread 28 of a drive shaft 30. The drive shaft 30 is coupled to a track (not shown) of the ring 16. Rotation of the output shaft 22, turns the drive shaft 30 and causes the entire cutting head assembly 18 to move about the cornea 14 along an arcuate path.

The output shaft: 22 also has a pin 32 that extends into a corresponding slot 34 of a blade holder 36. The blade holder 36 is attached to a blade 38 which has a cutting edge 40 that cuts the cornea 14. Rotation of the output shaft 22 causes a reciprocating transverse movement of the blade 38. The reciprocating movement of the blade 38 cuts corneal tissue while the drive shaft 30 moves the entire assembly 18 across the cornea 14. The blade assembly 10 can be replaced by removing the assembly 10 from a blade cavity 42 of the cutting head assembly 18.

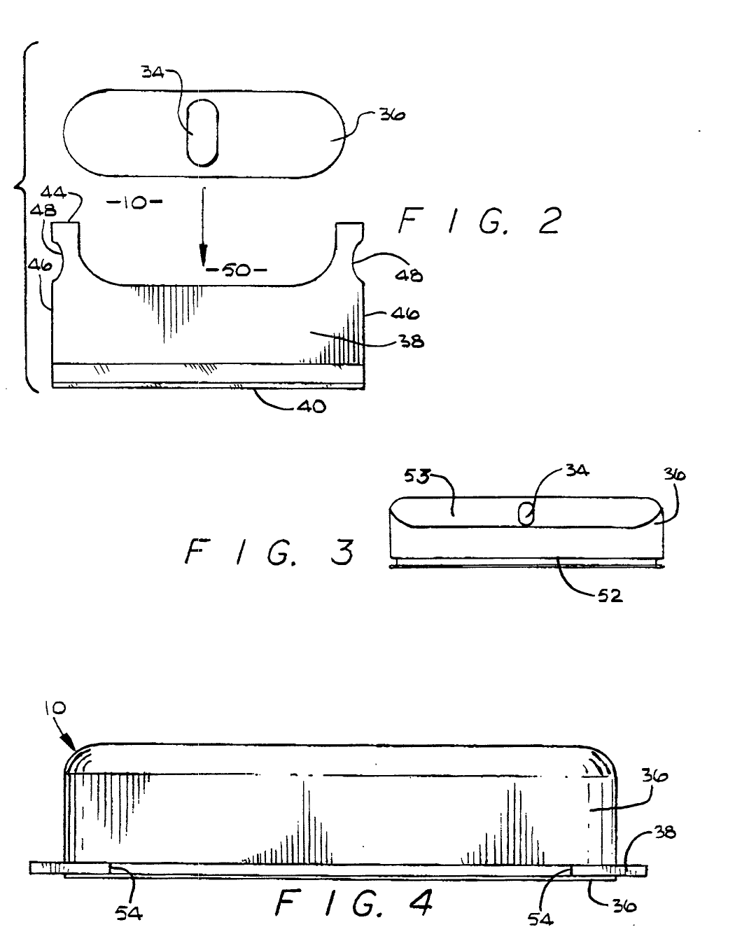

FIG. 2 shows an embodiment of a blade assembly 10 that includes the blade holder 36 and a blade 38. The blade 38 is typically constructed from a hard stainless steel material that is stamped or machined into the configuration shown. The blade 38 may include the cutting edge 40, a rear edge 44 and a pair of side edges 46. The side edges 46 may each have a notch 48. The rear edge 44 may also have a notch 50.

The notches 48 may provide a feature that allows an operator to grab the blade assembly 10 and load the assembly 10 into the microkeratome 12. Additionally, a plurality of blades 38 may be loaded and transported on a rack (not shown) with pins that extend through the notches 48. The notches 48 may also provide reference surfaces for fixture alignment pins (not shown) used to align and calibrate the blade holder 36 with the blade 38.

As shown in FIG. 3, the blade holder 36 may have an outer groove 52. The blade holder 36 may also have a tapered top surface 53 to provide clearance for the pin 32 when the assembly 10 is loaded into the microkeratome 12. The blade holder 36 may be constructed from a plastic material, wherein the groove 52 and slot 34 are either molded or machined into the holder 36. Referring to FIG. 2, the blade holder 36 can be assembled onto the blade 38 by pushing the holder 36 into the notch 50, so that the edge of the notch 50 extends into the groove 52 of the side of the blade holder 36.

As shown in FIG. 4, the blade holder 36 engages the inner edges 54 of the blade notch 52. The blade holder 36 is held in place by frictional forces between the holder 36 and the edges 54 to create a frictional fit. The blade holder 36 may be further secured to the blade 38 by an adhesive or other means.

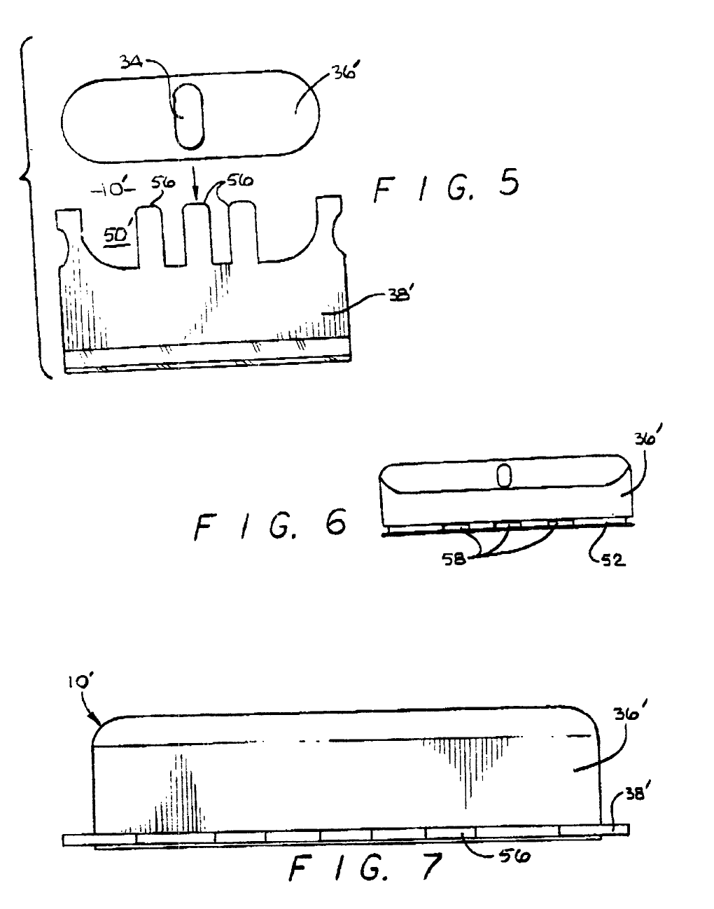

FIGS. 5, 6 and 7 show another embodiment of a blade assembly 10′. In this embodiment, the blade 38′ has one or more fingers 56 within the notch 50′. The fingers 56 can extend into corresponding slots 58 of the blade holder 36′. The finger(s) 56 increase the surface area and corresponding frictional forces that couple and lock the blade 38′ to the blade holder 36′.

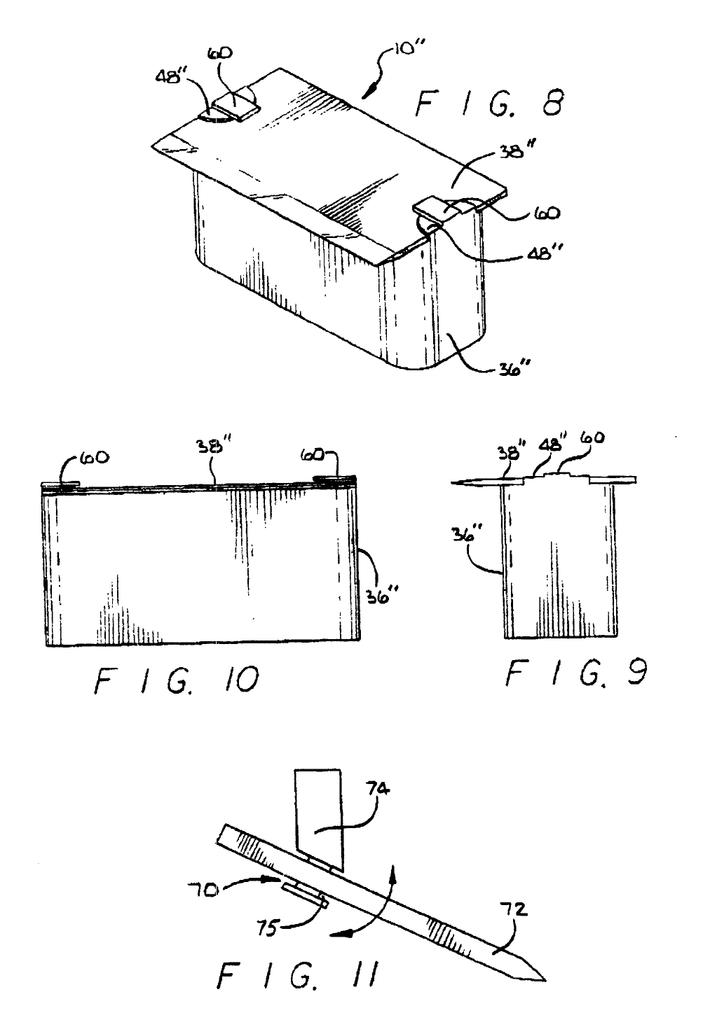

FIGS. 8, 9 and 10 show yet another embodiment of a blade assembly 10″. The blade holder 36″ of the assembly 10″ has a pair of clips 60 that secure the holder 36″ to the blade 38″ within blade notches 48″. The clips 60 secure the holder 36″ to the blade 38″ with frictional forces. With this embodiment the blade holder 36″ can move relative to the blade 38″ during installation into the microkeratome 12. The relative movement provides a mechanical float feature that compensates for tolerances in the cutting head assembly 18, particularly the cavity 42 of the microkeratome.

FIG. 11 shows another embodiment of a blade holder assembly 70 wherein a blade 72 can pivot relative to the blade holder 74 as indicated by the arrow. This embodiment provides a mechanical float that will compensate for tolerances in the assembly 20 and the microkeratome 12. The float is created by a gap 75 between the blade holder 74 and the blade 72. The blade holder 74 may be held in place by frictional forces between an outer edge of the holder 74 and an inner edge of the blade 72.

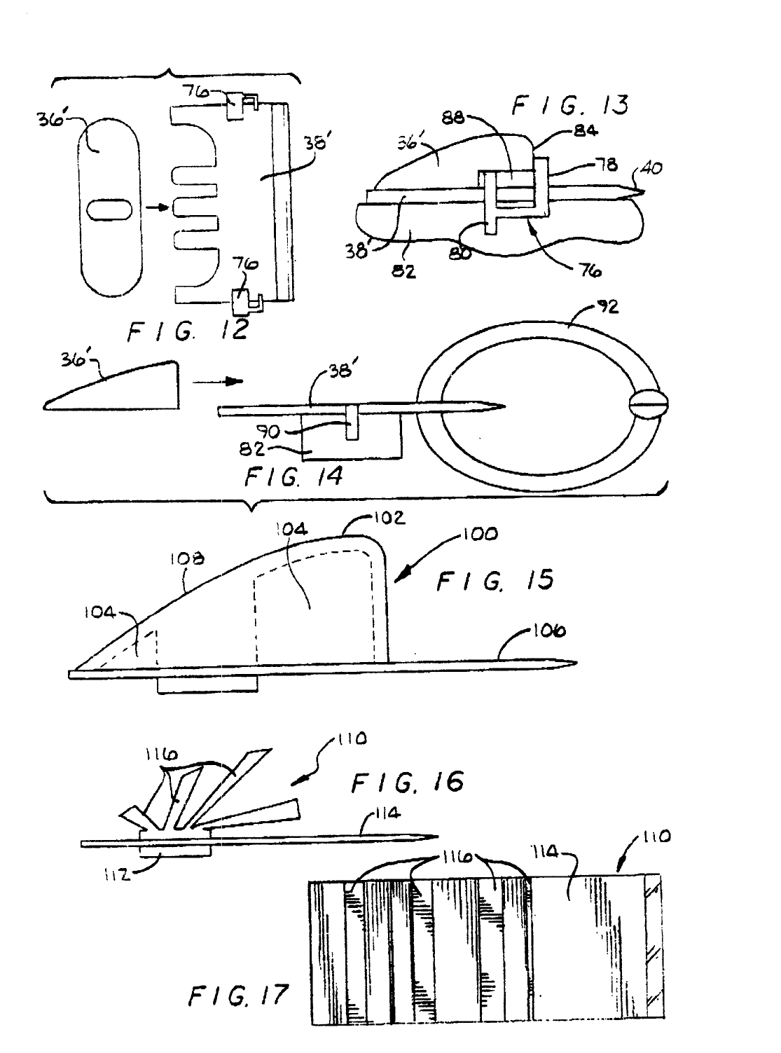

FIGS. 12 and 13 show a method for assembling and calibrating the blade holder 36′ to the blade 38′. The blade 38′ may be held in place by a pair of stabilizer posts 76. The posts 76 extend through the notches 48 of the blade 38. Each stabilizer post 76 includes a stop 78 that is connected to a pin 80. Each pin 80 is attached to a fixture plate 82.

The blade holder 36′ is pushed onto the blade 38′ until a reference surface 84 of the holder 36′ abuts against the stop 78. The reference surface 84 rest against a corresponding reference surface 86 of the cutting head assembly 18 shown in FIG. 1. The stop 78 provides a datum point that closely controls the distance between the reference surface 84 and the cutting edge 40 of the blade 38. The distance between the reference surface 84 and the cutting edge 40 defines the cutting depth of the blade 38′. The blade holder 36′ may have a pair of outer notches 88 that provide a clearance for the pins 80 when the holder 36′ is pushed onto the blade 42.

FIG. 14 shows another means for assembling and calibrating the blade holder 36′ to the blade 38′. The blade 38′ can be secured to a fixture plate 82 by a couple of pins 90 that extend into the blade notches. A clamp 92 is then coupled to the blade 38. The blade holder 36′ is pushed onto the blade 38′ until the reference surface 84 abuts against the clamp 92. The distance between the clamp 88 and the cutting edge 40 can be accurately controlled to minimize the tolerance between the reference surface 84 and the edge 40.

FIG. 15 shows another embodiment of a blade assembly 100 that includes a blade holder 102 which has one or more cavities 104. The blade holder 102 is coupled to a blade 106 by any of the embodiments shown in FIGS. 2-11. The cavities 104 reduce the stiffness of the blade holder 104 so that the holder 104 can be more readily installed into an undersized blade cavity 42. Additionally, a tool (not shown) can be inserted in a cavity 104 and used to push the blade holder 102 onto the blade 106. The blade holder 102 may also have a contoured top surface 108 that reduces the surface area in contact with the cutting head assembly 18. The contoured surface 108 reduces the tolerance requirements of the holder 102 and the cavity 42.

FIGS. 16 and 17 show another embodiment of a blade assembly 110 that includes a blade holder 112 coupled to a blade 114. The blade holder 112 can be attached to the blade 114 by an interference fit as described in FIGS. 2-11. The blade holder 112 includes a plurality of fingers 116. The fingers 116 provide a means to grasp the assembly 110. The individual fingers 116 also minimize the friction and lack of fit with the blade cavity 42. The most distal finger 116 provides a reference surface that abuts against the corresponding reference surface of the cavity 42.

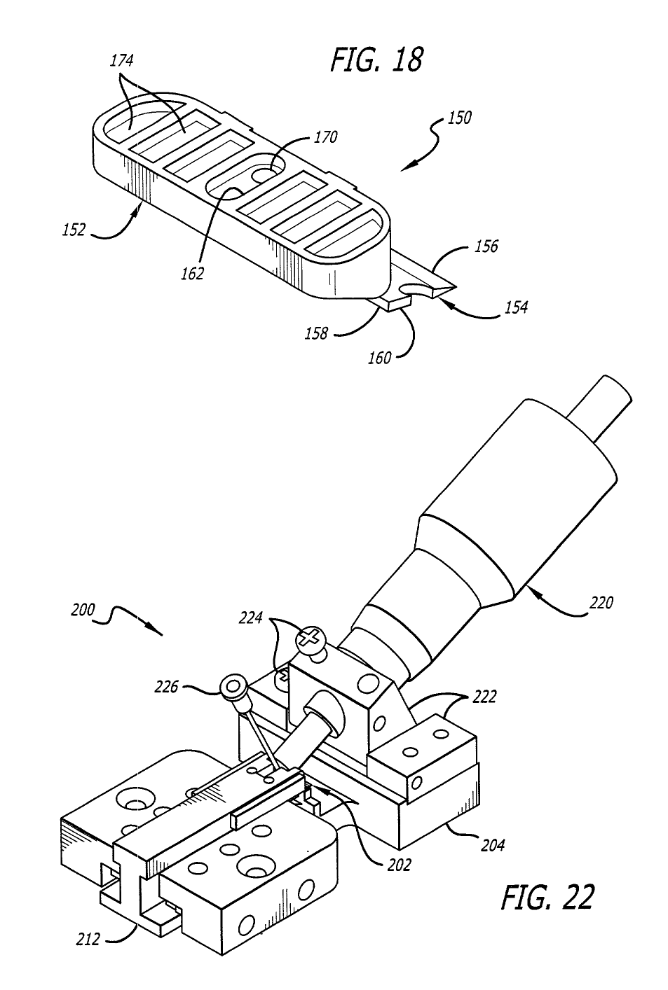

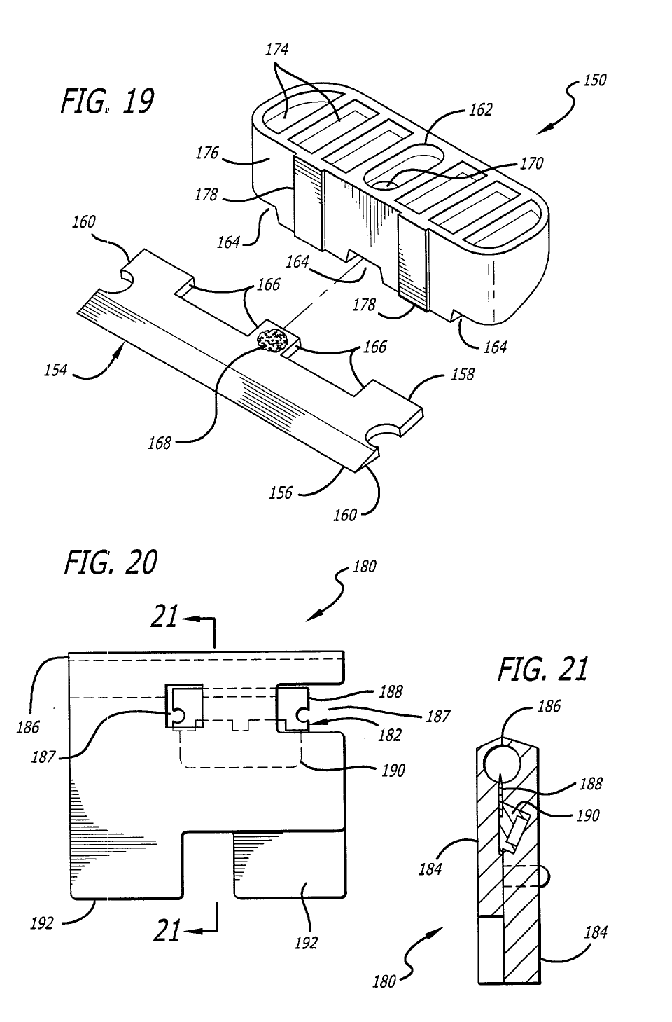

FIGS. 18 and 19 show another embodiment of a blade assembly 150. The assembly 150 includes a blade holder 152 that is attached to a blade 154. The blade 154 has a cutting edge 156, a rear edge 158 and a pair of side edges 160. The blade holder 152 may have a recess 162 that can receive an output pin (not shown) of a drive assembly (not shown).

The blade holder 152 may have a pair of slots 164 that are pressed into corresponding tabs 166 of the blade 154. The blade holder 152 can be bonded to the blade 154 by a bonding agent 168 introduced through a hole 170 in the recess 162.

The blade holder 152 may be constructed from a molded plastic material. Constructing the blade holder 152 as a solid rectangular block may result in warpage of the holder 152 after the molding process. To minimize warpage the blade holder 152 may have a plurality of cavities 174 that relieve the residual stresses in the plastic.

The blade holder 152 has a front surface 176 that presses against a corresponding surface of a microkeratome (not shown). The distance from the front surface 176 to the cutting edge of the blade 154 defines the cutting depth of the blade 154 into a cornea. The molding process may create a wavy or otherwise irregular front surface 176 that varies the cutting depth of the blade 154. To minimize surface irregularities, the blade holder 152 may have a pair of raised surfaces 178 that extend from the front surface 176. The raised surfaces 178 have a smaller area and thus are less likely to have a wavy or otherwise irregular surface. The raised surfaces 178 make contact with the microkeratome and together provide a reference surface that accurately controls the cutting depth of the blade 154.

Blade assemblies of the prior art allow the rear edge of the blade 154 to be exposed. When cutting a cornea the metal rear edge may strike the ring of the microkeratome. Contact between the ring and blade may cause wear in the ring and undesirable metal filings. To prevent metal to metal contact the plastic blade holder 152 extends beyond the rear edge of the blade 154. Any contact between the blade assembly 150 and the metal microkeratome ring is with the plastic blade holder, thereby eliminating wear of the ring and the creation of metal filings.

The blade holder 152 may have a color that corresponds to the cutting depth of the blade 154. Each color would correspond to a specific cutting depth. For example, a blue colored blade holder 152 may indicate that the cutting depth of the blade is 160 microns. A red blade holder 152 may indicate that the cutting depth of the blade 154 is 180 microns. Color coding the blade holder 152 allows the surgeon to quickly identify the cutting depth of the blade assembly 150 before assembly into a microkeratome. Although a color coding scheme has been described, it is to be understood that other visual indicators may be employed to convey the cutting depth of the blade assembly 150.

FIGS. 20 and 21 show a package 180 that can be used to carry and transport a blade assembly 182. The package 180 may have a pair of covers 184 that can rotate about a hinge 186. The entire package 180 can be constructed from a molded plastic material. The package 180 may have openings 187 to allow visual inspection of the blade 188 and blade holder 190. By way of example, an optical pattern recognition machine (not shown) may be used to measure the cutting depth of the blade 188 through the openings 187. Each cover 184 may have a tab 192 that can be pulled to open the package 180.

The package 180 may be color coded to provide a visual indication of the blade cutting depth. Although color coding is described, it is to be understood that the package 180 may incorporate other visual indicators to provide an indication of the cutting depth.

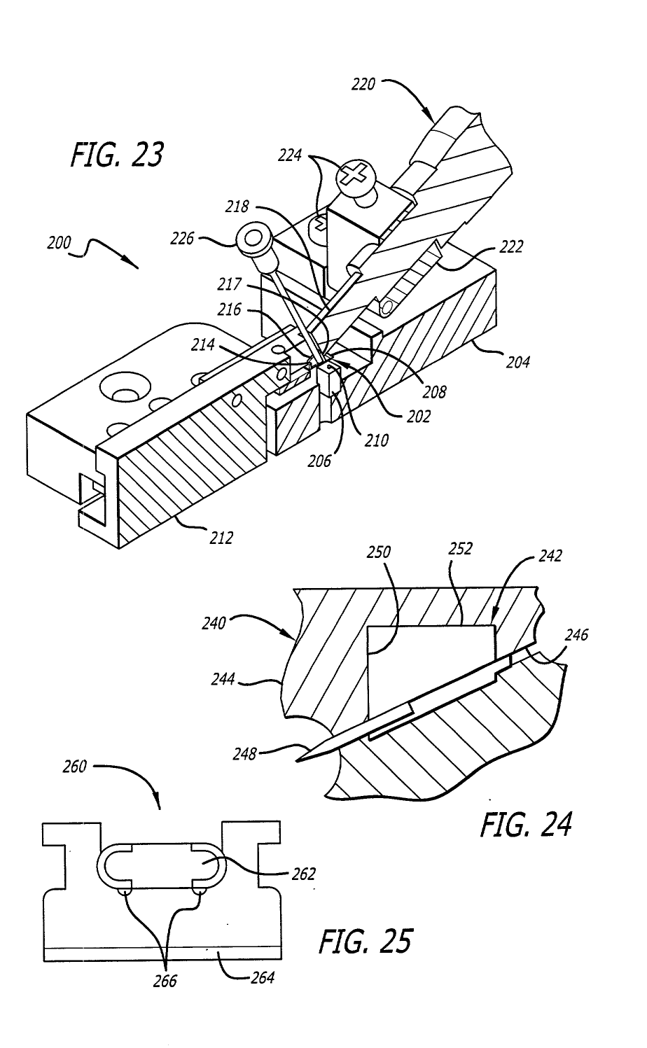

FIGS. 22 and 23 show a tooling assembly 200 for assembling a blade assembly 202. The assembly 200 includes a base 204 with a blade support bar 206 that can support a blade 208. The support bar 206 may have a pair of pins 210 that extend through the notches located on the sides of the blade 208. The pins 210 and notches align the blade 208 within the tool 200.

The tool assembly 200 may further have a slide bar 212 that can push a blade holder 214 onto the blade 208. The slide bar 212 may have a notch 216 that corresponds to the outer profile of the blade holder 214. The slide bar 212 can be manually actuated, or automatically actuated by a motor, solenoid, or other means.

The slide bar 212 pushes the blade holder 214 onto the blade 208 until a front surface 217 of the holder 214 engages an adjustable stop 218. The adjustable stop 218 may be the tip of a micrometer 220 that can be moved relative to the base 204. Actuation of the micrometer 220 moves the adjustable stop 218 and varies the cutting depth of the blade assembly 202. The tool assembly 200 thus allows a manufacturer to accurately vary the cutting depth of each blade assembly 202. The micrometer 220 can be coupled to the base 204 by a plurality of blocks 222 and alignment screws 224. The micrometer may be manually or automatically actuated.

The tool assembly 200 may further include a cannula 226. The slide bar 212 may include access for the cannula 226 to reach the hole 170. The cannula 226 may allow a bonding agent to be applied to the blade 208 during the assembly process. The cannula 226 has a location which does not interfere with the pressing operation of the tool assembly 200.

FIG. 24 shows a gauge 240 that can be used to determine whether a blade assembly 242 is within acceptable manufacturing tolerance limits. The gauge 240 includes a housing 244 that has a slot 246 adapted to receive a blade 248. The housing 244 further has a cavity 250 adapted to receive a blade holder 252. The cavity 250 may have the minimum or maximum dimensions allowed for the blade holder.

There are typically two different gauges 240 used to check the tolerances of the blade assembly 242, a minimum gauge and a maximum gauge. The minimum gauge contains the smallest cavity allowable. If a blade holder fits within the minimum gauge then the holder is too small and is rejected. The maximum gauge has the largest cavity allowable. If a blade holder does not fit within the maximum gauge then the holder is too large.

FIG. 25 shows another embodiment of a blade assembly 260 that includes a blade holder 262 attached to a blade 264. The blade 264 may include a pair of openings 266 that provide reference points for an optical inspection machine.

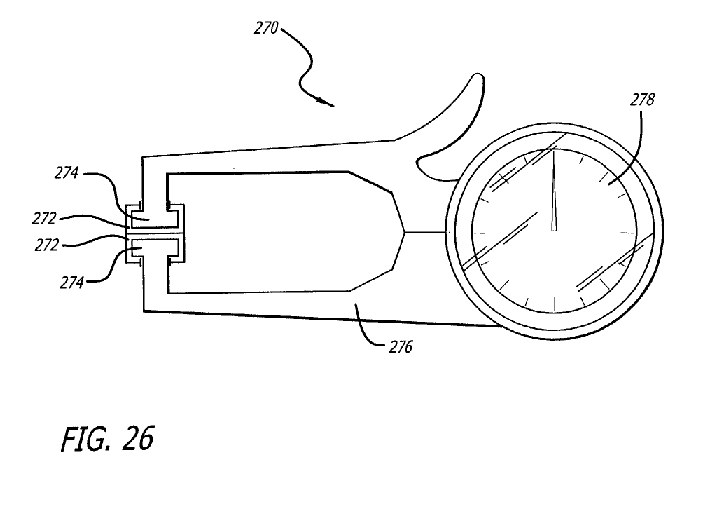

FIG. 26 shows a caliper assembly 270 for measuring the thickness of a corneal flap. The assembly 270 includes a pair of protective covers 272 that are attached to tips 274 of a caliper 276. The covers 272 may be constructed from a plastic material that can be disposed after each measurement. The caliper 276 has a dial 278 that provides a readout. A flap can be measured by attaching the covers 272 to the tips 274. The tips 274 are closed to measure the thickness of the covers 272. The caliper 276 is then opened and closed about a flap. The thickness of the covers 272 is subtracted from the readout to provide the thickness of the flap.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

What is claimed is:

1. A blade assembly that can be assembled into a medical device used to cut a cornea, comprising:
a blade that has a cutting edge, a rear edge, and a pair of side edges that extend between said cutting edge and said rear edge; and,
a blade holder that is coupled to said blade to define a cutting depth, said blade holder having a color that corresponds to said cutting depth of said blade.

2. The blade assembly of claim 1, wherein said cutting depth is dependent upon a dimension from a front surface of said blade holder and said cutting edge of said blade.

3. The blade assembly of claim 2, wherein said front surface includes a raised surface.

4. The blade assembly of claim 1, wherein said blade holder includes a recess and a plurality of cavities.

5. The blade assembly of claim 1, wherein said blade holder has a hole that receives a bonding agent that bonds said blade holder to said blade.

6. The blade assembly of claim 1, wherein said blade holder extends from said rear edge of said blade.

7. A blade assembly that can be assembled into a medical device used to cut a cornea, comprising:
a blade that has a cutting edge, a rear edge, and a pair of side edges that extend between said cutting edge and said rear edge; and,
a blade holder that is coupled to said blade to define a cutting depth, said blade holder having indicator means for providing an indication of said cutting depth of said blade.

8. The blade assembly of claim 7, wherein said cutting depth is dependent upon a dimension from a front surface of said blade holder and said cutting edge of said blade.

9. The blade assembly of claim 8, wherein said front surface includes a raised surface.

10. The blade assembly of claim 7, wherein said blade holder includes a recess and a plurality of cavities.

11. The blade assembly of claim 7, wherein said blade holder has a hole that receives a bonding agent that bonds said blade holder to said blade.

12. The blade assembly of claim 7, wherein said blade holder extends from said rear edge of said blade.

13. A blade assembly that can be assembled into a medical device used to cut a cornea, comprising:
a blade that has a cutting edge, a rear edge, and a pair of side edges that extend between said cutting edge and said rear edge; and,
a blade holder that is coupled to said blade, said blade having a recess and a plurality of cavities.

14. The blade assembly of claim 13, wherein said blade holder has a front surface that includes a raised surface.

15. The blade assembly of claim 13, wherein said blade holder has a hole that receives a bonding agent that bonds said blade holder to said blade.

16. The blade assembly of claim 13, wherein said blade holder extends from said rear edge of said blade.

17. A blade assembly that can be assembled into a medical device used to cut a cornea, comprising:
a blade that has a cutting edge, a rear edge, and a pair of side edges that extend between said cutting edge and said rear edge; and,
a blade holder that is coupled to said blade, said blade holder having a recess and cavity means.

18. The blade assembly of claim 17, wherein said blade holder has a front surface that includes a raised surface.

19. The blade assembly of claim 17, wherein said blade holder has a hole that receives a bonding agent that bonds said blade holder to said blade.

20. The blade assembly of claim 17, wherein said blade holder extends from said rear edge of said blade.

21. A blade assembly that can be assembled into a medical device used to cut a cornea, comprising:
a blade that has a cutting edge, a rear edge, and a pair of side edges that extend between said cutting edge and said rear edge;
a blade holder that is coupled to said blade, said blade having a recess and a hole; and,
a bonding agent that attaches said blade holder to said blade.

22. The blade assembly of claim 21, wherein said blade holder has a front surface that includes a raised surface.

23. The blade assembly of claim 21, wherein said blade holder extends from said rear edge of said blade.

24. A blade assembly that can be assembled into a medical device used to cut a cornea, comprising:
a blade that has a cutting edge, a rear edge, and a pair of side edges that extend between said cutting edge and said rear edge; and,
a blade holder that is coupled to said blade, said blade holder having a recess and bonding access means; and,
bonding means for bonding said blade holder to said blade.

25. The blade assembly of claim 24, wherein said blade holder has a front surface that includes a raised surface.

26. The blade assembly of claim 24, wherein said blade holder extends from said rear edge of said blade.

27. A blade assembly that can be assembled into a medical device used to cut a cornea, comprising:
a blade that has a cutting edge, a rear edge, and a pair of side edges that extend between said cutting edge and said rear edge; and,
a blade holder that is coupled to said blade, said blade having a front surface that includes a raised surface.

28. The blade assembly of claim 27, wherein said blade holder extends from said rear edge of said blade.

29. A blade assembly that can be assembled into a medical device used to cut a cornea, comprising:
a blade that has a cutting edge, a rear edge, and a pair of side edges that extend between said cutting edge and said rear edge; and,
a blade holder that is coupled to said blade, said blade holder having a front surface and reference surface means for establishing a cutting depth of said blade.

30. The blade assembly of claim 29, wherein said blade holder extends from said rear edge of said blade.

31. A blade assembly that can be assembled into a medical device used to cut a cornea, comprising:
a blade that has a cutting edge, a rear edge, and a pair of side edges that extend between said cutting edge and said rear edge; and,
a blade holder that is coupled to said blade such that said blade holder extends from said rear edge of said blade.

32. An assembly tool for assembling a blade holder to a blade to create a blade assembly used to cut a cornea, comprising:
a base;
a slide bar coupled to said base; and,
an adjustable stop that is coupled to said base.

33. The assembly tool of claim 32, further comprising a cannula coupled to said base.

34. The assembly tool of claim 32, wherein said adjustable stop includes a micrometer.

35. The assembly tool of claim 32, further comprising a pin attached to said base.

36. An assembly tool for assembling a blade holder to a blade to create a blade assembly used to cut a cornea, comprising:
base means to support a blade;
press means for pressing a blade holder into the blade; and,
adjustment means for varying a cutting depth of the blade.

37. The assembly tool of claim 36, further comprising bonding means for introducing a bonding agent to the blade to bond the blade holder to the blade.

38. The assembly tool of claim 36, wherein said adjustment means includes a micrometer.

39. The assembly tool of claim 36, further comprising alignment means attached to said base.

40. A method for assembling a blade assembly, comprising;
adjusting a position of a stop; and,
pushing a blade holder onto a blade until the blade holder engages the stop.

41. A blade package, comprising:
a pair of covers, at least one of said covers having an opening to allow inspection of the blade assembly.

42. A blade package, comprising:
a pair of covers, at least one cover having a color indicative of a cutting depth of the blade assembly.

43. A blade package, comprising:
a pair of covers that enclose a blade assembly, at least one cover having means for providing an indication of the cutting depth of the blade assembly.

44. A gauge for a blade assembly, comprising:
a housing that has a slot adapted to receive a blade and a cavity adapted to receive a blade holder attached to the blade.

45. A blade assembly that can be assembled into a medical device used to cut a cornea, comprising;
a blade holder having a front surface; and
a blade attached to said blade holder, said blade having a pair of side edges, a cutting edge, a rear edge and an opening located between said cutting edge and said front surface between said side edges.

46. A caliper assembly for measuring a corneal flap, comprising:
a caliper that has a readout and a tip; and,
a cover attached to said tip.

47. A method for measuring a corneal flap, comprising:
attaching a pair of covers to a pair of tips of a caliper;
measuring a combined thickness of the covers;
reading a measurement of a corneal flap located between the cover; and,
determining the thickness of the corneal flap by subtracting the thicknesses of the covers from the reading.
